(19) United States
(12) Patent Application Publication
(10) Pub. No.: US 2025/0205178 A1
(43) Pub. Date: Jun. 26, 2025

(54) METHODS AND COMPOSITIONS FOR INHIBITING VIRAL METHYLTRANSFERASES

(71) Applicant: ARIZONA BOARD OF REGENTS ON BEHALF OF THE UNIVERSITY OF ARIZONA, Tucson, AZ (US)

(72) Inventors: Hongmin Li, Tucson, AZ (US); Zhong Li, Tucson, AZ (US)

(21) Appl. No.: 19/074,850

(22) Filed: Mar. 10, 2025

Related U.S. Application Data
(63) Continuation of application No. PCT/US23/73683, filed on Sep. 7, 2023.
(60) Provisional application No. 63/374,939, filed on Sep. 8, 2022.

(51) Int. Cl. A61K 31/12; A61K 31/706; A61P 31/14; C12Q 1/48

(52) U.S. Cl. CPC A61K 31/12; A61K 31/706; A61P 31/14; C12Q 1/48; G01N 2333/91011

(57) ABSTRACT

SARS-CoV-2 has caused a global pandemic with significant humanity and economic loss since the beginning of 2020. Flaviviruses such as Zika and Dengue viruses are also significant human pathogens. Although SARS-CoV-2 vaccines are effective in preventing severe disease outcomes, they are less effective in controlling infection or re-infection, particularly due to rapid evolution of viral variants of SARS-CoV-2. Currently only limited options are available to treat SARS-CoV-2 and flavivirus infections for vulnerable populations. Potential of a future pandemic of other viruses is high. The present invention features compositions and methods for a universal high throughput screening (HTS) assay to identify inhibitors targeting the S-adenosyl-L-methionine (SAM)-binding site of viral methyltransferases (MTases) using SAM as a methyl donor.

### CROSS-REFERENCE TO RELATED APPLICATIONS

This application is a continuation and claims benefit of International Application No. PCT/US2023/073683 filed Sep. 7, 2023, which claims benefit of U.S. Provisional Application No. 63/374,939 filed Sep. 8, 2022, the specification of which is/are incorporated herein in their entirety by reference.

### STATEMENT REGARDING FEDERALLY SPONSORED RESEARCH OR DEVELOPMENT

This invention was made with government support under Grant No. AI175435 awarded by National Institutes of Health. The government has certain rights in the invention.

### FIELD OF THE INVENTION

The present invention features methods and compositions for inhibiting viral methyltransferases, specifically for the treatment of a viral infection (e.g., SARS-CoV-2 and flaviviruses).

### BACKGROUND OF THE INVENTION

### HTS Assay

The COVID-19 pandemic has lasted for more than two years. The aetiological coronavirus (CoV), SARS-CoV-2, was first reported in Wuhan, China in December 2019 and has spread to more than 200 countries and territories. It has resulted in more than 500 million infections and 6 million deaths according to the World Health Organization. Many SARS-CoV-2 variants with varying infection rates and lethality have evolved during this period. So far, only a few small molecule drugs, including remdesivir, Paxlovid and molnupiravir, have been approved by U.S. Food and Drug Administration (FDA) for the treatment or Emergency Use Authorization of COVID-19. Both remdesivir and molnupiravir target the viral RNA-dependent RNA polymerase NSP12, whereas Paxlovid inhibits the viral main protease NSP5. In addition to drugs, COVID-19 vaccines have been developed at an amazing pace, which have helped reduce the death rate substantially. However, vaccination does not prevent people from infection or reinfection of rapidly developing variants of SARS-CoV-2. There is an urgent need for the development of small molecule drugs for unvaccinated individuals and people who are infected despite being vaccinated.

The SARS-CoV-2 enzymes are potential targets for small molecule drug discovery. SARS-CoV-2 is a single-stranded positive sense RNA virus with large genome size (˜30 kb). It encodes 4 structural, 16 non-structural (NSP) and 6 accessory proteins. SARS-CoV-2 has a type I cap at the 5′ end. A crucial step in the viral replication pathway is the RNA 5′-capping, a conserved biochemical mechanism in CoVs and in many other viruses such as (+)ssRNA flaviviruses, dsDNA poxviruses, dsRNA reoviruses, and (−)RNA viruses such as vesicular stomatitis virus. For CoVs, this capping process involves four steps. In step (1) the y phosphate is removed from the nascent 5′-triphosphorylated RNA (5′-pppRNA) to produce a 5′-diphosphorylated RNA (5′-ppRNA) by an RNA triphosphatase (RTPase). In step (2), a GMP moiety is transferred from GTP to the 5′-ppRNA by a guanylyltransferase (GTase) to form the core cap structure GpppN-RNA. In step (3), a guanine-N7-methyltransferase (N7-MTase) methylates the cap guanine at the N7 position. In the final step (4), a nucleoside-2′-O-MTase (2′-O-MTase) methylates the ribose-2′-OH position of the first nucleotide of the RNA.

All viral MTases use S-adenosylmethionine (SAM) as methyl donor and generate S-adenosyl-L-homocysteine (SAH) as a by-product. SARS-CoV-2 encodes two different proteins-NSP14 and NSP16—to perform the N7- and 2′-O MTase activities in steps (3) and (4), respectively. NSP14 is a bifunctional enzyme with an N-terminal 3′→5′ exoribonuclease (ExoN) domain and a C-terminal N7 MTase domain, whereas NSP16 only has 2′-O MTase function. Both NSP14 and NSP16 form complexes with co-factor protein NSP10. However, NSP10 is only essential for the 2′-O MTase activity of NSP16 and the ExoN activity of NSP14 but not for the N7 MTase activity of NSP14. The NSP14 MTase plays an essential role in viral RNA 5′-capping by methylating viral RNA at the N7 position of the 5′ guanine cap. The N7-MTase activity facilitates viral mRNA stability, viral translation initiation and is essential for viral replication.

MTases are validated drug targets. Identification of potent inhibitors against viral MTases has been hampered by a lack of proper chemical probes to develop high throughput screen (HTS) assays. Most existing MTase function assays rely on radioactive materials such as ³²P-labelled RNA substrate, which is not suitable for HTS. In addition, several HTS assays were developed using non-radioactive RNA substrates. However, they have various limitations. Pearson et al. reported a mass spectrometry (MS)-based HTS assay to identify inhibitors for the NSP14 MTase. The assay requires a specific RapidFire MS instrument which is not widely available for many academic and industrial laboratories. HTS assays were also developed based on homogenous time-resolved fluorescence (HTRFR®). Although the HTRF assay does not use radioactive materials, the high cost of the commercial EPIgeneous Methyltransferase Kit (Cisbio, MA) prohibits large-scale HTS for academic institutions. Another limitation is that any assay based on the MTase enzyme activity will require generation of a large quantity of capped viral RNA substrates. However, generation of large quantity enough for large-scale primary HTS screen is prohibitive because available assays will use micromolar concentration of the RNA substrates in a reaction. Interestingly, Kasprzyk et al. reported a functional Py-FLINT MTase HTS assay based on a pyrene-labelled GpppA fluorescent substrate probe. Although the Py-FLINT assay is suitable for large-scale HTS, the assay may only be limited to a few viral MTases that can use the GpppA dinucleotide as a substrate. It is reported that many viral MTases including flavivirus and SARS-CoV-2 NSP16/10 MTases cannot methylate the GpppA dinucleotide.

### Compositions.

Viruses within the arthropod-borne flavivirus genus are more than 70 related arthropod-borne viruses, such as Zika virus (ZIKV), West Nile virus (WNV), Japanese encephalitis virus (JEV), and dengue virus (DENV). Four serotypes of DENV, DENV 1-4, are responsible for sickness ranging from mild dengue fever with flu-like symptoms to hemorrhagic fever and dengue shock syndrome, which can be fatal. WNV and JEV can all cause fatal complications such as meningitis and encephalitis. Similarly, ZIKV is an arthropod-borne virus that can cause serious brain deformities in fetuses, including microcephaly and other birth defects. It can also be transmitted sexually and by blood transfusion

There are effective vaccines for yellow fever virus (YFV), JEV, and tick-borne encephalitis virus (TBEV), but there is no vaccine for WNV or ZIKV. A DENV vaccine was recently approved in a few countries including the United States. However, it is not effective for young children, and posts increased risk in naive children. In addition, a DENV vaccine, TKEDA's QDENGA, also known as TAK-003, has been authorized by Brazil's National Health Surveillance Agency for individuals between the ages of 4 and 60 to prevent dengue caused by any of the four virus serotypes. Additionally, mass vaccination presents several challenges. As a result, therapeutic treatments that disrupt crucial phases of the flavivirus life cycle must be developed.

The genome of single-stranded positive-sense flaviviral RNA is approx. 11 kb in length and has a single open reading frame (ORF) flanked by 5′ and 3′ untranslated regions. The viral single ORF encodes a polyprotein precursor that is processed into three structural proteins (capsid, premembrane or membrane, and envelope) and seven nonstructural (NS) proteins (NS1, NS2a, NS2b, NS3, NS4a, NS4b, and NS5) by the viral NS2b-NS3 protease complex and host proteases.

In eukaryotes, the 5′ end of mRNA is modified by addition of a 7-methylguanosine (m⁷G) cap. This alteration promotes translation, enhances the ability to distinguish between endogenous mRNAs and foreign RNA materials, and inhibits premature transcript breakdown. Similar to that of host mRNA, the 5′ end of the viral genome is capped by a type I m⁷G cap. In addition, the 2′-O positions of adenosine ribonucleotides of the viral genome are also methylated. The methylations are catalyzed by a highly conserved protein known as NS5 encoded by flavivirus.

NS5 has dual enzyme activities that regulate viral genome methylation and replication. The N-terminal domain of flavivirus NS5 has MTase activity, whereas the NS5 C-terminal domain is an RNA-dependent RNA polymerase. Flavivirus NS5 MTase catalyzes both methylations at the N7 position of the guanine cap and at the 2′-O position of the first transcribed ribonucleotide to create ₇^{Me}GpppA_{2′O}^{Me}-RNA. Recombinant MTases from various flaviviruses sequentially generate m⁷GpppAm-RNA using SAM as the methyl donor, resulting in methylated RNA and the byproduct S-adenosylhomocysteine (SAH). In addition, the ribose 2′-O of internal adenosines is also methylated by flavivirus NS5 MTase. These methylations are essential for the survival of the virus in infected animals and play a significant role in viral replication. Since N7 MTase activity is required for the translation of viral mRNA into proteins, as demonstrated by biochemical research and reverse genetic analyses, defects in the N7 MTase activity are lethal to the virus. Moreover, it is well known that 2′-O methylation is essential for a virus to evade the host immune system.

Viral MTases are attractive targets for antiviral development. Inhibition of viral N7 MTase has been shown to suppress viral replication. Several small-molecule inhibitors of flavivirus N7 MTase have been discovered. These inhibitors were found using different in vitro methods, such as large-scale library screening and rational drug design based on screening results or substrate structures.

Although progress has been made in developing inhibitors against viral MTases, this process is significantly delayed due to a lack of proper chemical probes to develop high throughput screen (HTS) assays. Traditional MTase functional assays use a radio-labeled RNA substrate, preventing it from development of HTS assays. In addition, although non-radioactive HTS assays were developed for a few viral MTases, they have various limitations. A mass spectrometry (MS)-based HTS assay was developed to identify inhibitors for the NSP14 MTase of SARS-CoV-2. However, a specific RapidFire MS instrument is required, making it less desirable. Nonradioactive homogeneous time-resolved fluorescence (HTRF) was also used to develop HTS assays. However, the commercial kit has several limitations. First, the EPIgeneous Methyltransferase Kit (Cisbio, MA), is very expensive, preventing it from being used in large-scale HTS by academic institutions. Second, a large quantity of capped viral RNA substrates will be required for any assay based on the MTase enzyme activity, including the HTRF kit. However, it is prohibitive to generate large quantities enough for large scale primary HTS screens. Recently, a functional Py-FLINT MTase HTS assay was reported using a pyrene-labeled GpppA fluorescent dinucleotide substrate probe. However, many viral MTases including flavivirus and SARS-CoV-2 NSP16/10 MTases cannot use the GpppA dinucleotide as a substrate, limiting the usage of the Py-FLINT assay.

The present invention is a fluorescence polarization (FP)-based HTS assay to identify inhibitors targeting the co-factor SAM-binding site of SARS-CoV-2 NSP14. In the assay, FP originated from an organic molecule, FL-NAH that targets the SAM binding site on NSP14. FL-NAH is fluorescent and structurally analogous to SAM and can be replaced by high-affinity compounds. Therefore, the assay could be applied for any SAM-dependent MTases such as SARS-CoV-2 NSP14 and NSP16, and flavivirus NS5 MTases. Using this assay, a small scale HTS was performed and identified two compounds, NSC 111552 and 288387, as candidate inhibitors of the NSP14 MTase. Inhibition of the MTase activity, binding affinity, antiviral efficacy and combination with known SARS-CoV-2 drugs were further investigated. These compounds are potent inhibitors of SARS-CoV-2 replication and can be further optimized for the development of structure-based inhibitors.

Furthermore, the present invention is a FP-based HTS assay to identify inhibitors targeting the cofactor SAM-binding site of flavivirus MTase. We used FL-NAH, a fluorescent analog of SAM, to develop a universal FP-based MTase assay. This assay is suitable for any SAM-dependent MTases, including the NS5 MTase of flavivirus. This assay allowed us to conduct a small scale HTS in which we discovered two compounds, NSC 111552 and 288387, as potential flavivirus MTase inhibitors. We further looked at the binding affinity, antiviral effectiveness, and inhibition of MTase activity by NSC 111552 and 288387. Compounds NSC 111552 and 288387 inhibit the MTases that possess an evolutionarily conserved SAM-binding pocket, which specifically interacts with the amino acids Val132, His110, Lys105, and Thr104 of the DENV3 MTase. These molecules have the potential to be further improved for the creation of structure-based inhibitors and are effective broad spectrum inhibitors of flaviviruses.

The present invention features methods and compositions for developing a universal fluorescence polarization (FP)-based HTS assay to identify inhibitors targeting the co-factor SAM-binding site of the SARS-CoV-2 NSP14, NSP16 and flavivirus NS5 MTases. In the assays described herein, FP originated from an organic molecule, FL-NAH, that targets the SAM binding site on SAM-dependent MTases such as NSP14, NSP16, and NS5. FL-NAH is structurally analogous to SAM and can be replaced by high affinity compounds. Therefore, the assay could be applied for any SAM-dependent MTases. Using this assay, a small scale HTS was performed and identified several compounds, NSC 111552, NSC288387, NSC70931, topotecan, and J006-1384, as candidate inhibitors of the NSP14, NSP16, and NS5 MTases. Inhibition of the MTase activity, binding affinity, and antiviral efficacy were further investigated. These compounds are potent inhibitors of SARS-CoV-2 and flavivirus replication and can be further optimized for the development of structure-based inhibitors.

### BRIEF SUMMARY OF THE INVENTION

It is an objective of the present invention to provide compositions and methods that allow for the inhibition of viral methyltransferases, as specified in the independent claims. Embodiments of the invention are given in the dependent claims. Still, the invention can be freely combined with each other if they are not mutually exclusive.

In some embodiments, the present invention features a fluorescence polarization (FP)-based method to identify an inhibitor targeting an S-adenosyl-L-methionine (SAM)-binding site of a viral methyltransferase (MTase) for a coronavirus or a flavivirus, said method comprising: (1) introducing the inhibitor and a fluorescent analog of a methyl donor SAM to the coronavirus or the flavivirus for them to compete to combine with the viral MTase of the coronavirus or the flavivirus; and (2) measuring binding of the fluorescent analog and the viral MTase, wherein binding of the fluorescent analog and the viral MTase increases the fluorescence polarization of the fluorescent analog, wherein binding of the inhibitor and the viral MTase reduces the fluorescence polarization of the fluorescent analog.

In some embodiments, the present invention features a composition comprising at least one compound selected from the following:

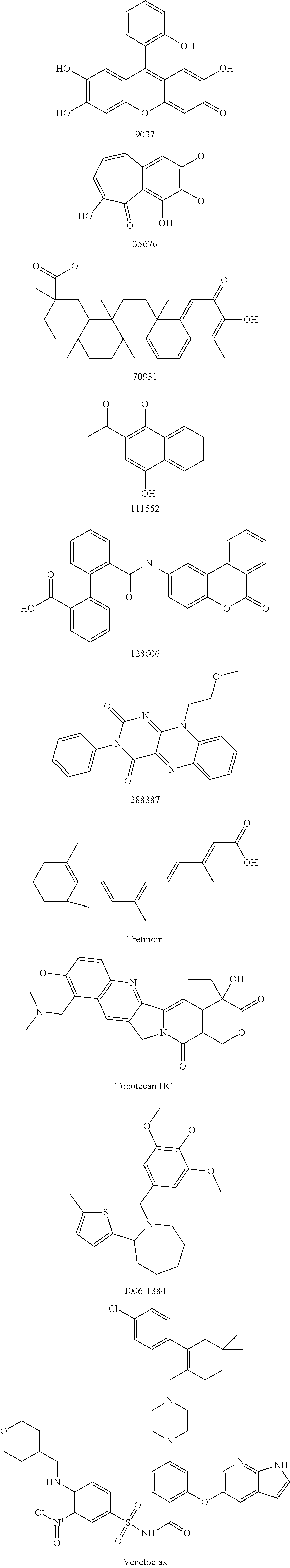

In some embodiments, the present invention features a method of treating a viral infection in a subject in need thereof, the method comprising administering to the subject a therapeutically effective amount of a composition that targets an S-adenosyl-L-methionine (SAM)-binding site of viral methyltransferases (MTases) for a coronavirus or a flavivirus using a SAM as a methyl donor. In other embodiments, the present invention features a composition for use in treating a viral infection in a subject in need thereof. In some embodiments, the composition may comprise at least one compound selected from the following:

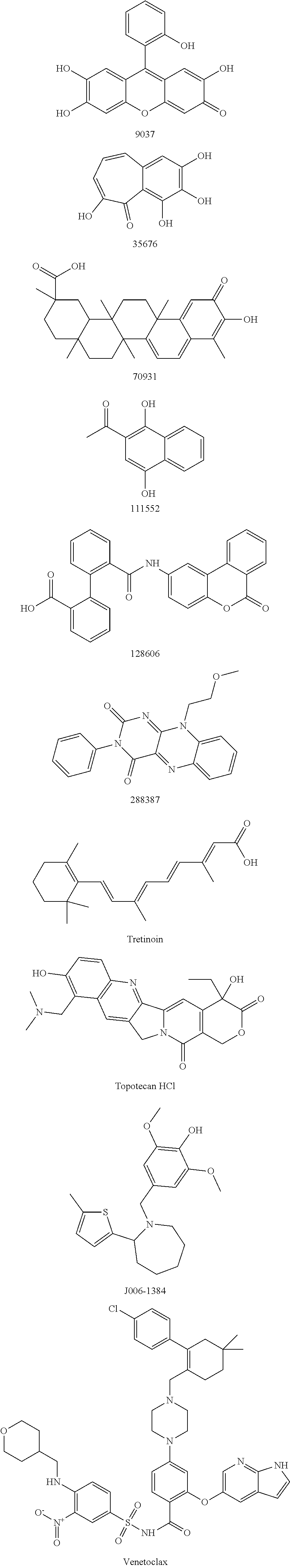

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWING(S)

The features and advantages of the present invention will become apparent from a consideration of the following detailed description presented in connection with the accompanying drawings in which:

FIG. 1 shows a dose-dependent FL-NAH FP assay. FL-NAH (50 nM) was applied to 2-fold diluted concentration series of NSP14 and NSP14-NSP10 complex. FP was calculated by measuring the parallel and perpendicular fluorescence with excitation and emission wavelengths of 485 nm and 528 nm, respectively. N=3

FIG. 2A shows SAH (25 μM, 31.25 μM, or 50 μM) inhibited FL-NAH (50 nM or 30 nM) binding to the SARS-CoV-2 NSP14 (0.5 μM), ZIKV NS5 (2.5 μM), or SARS-CoV-2 NSP16 (0.5 μM) MTases in 96-well plate. ****, p<0.0001.

FIG. 2B shows structures of NSC 111552 and 288387.

FIG. 2C shows dose-dependent inhibition of FL-NAH binding to the NSP14 MTase by NSC 111552 and 288387. N=3. FP values in the presence of compounds were normalized to that of the DMSO control (100%).

FIG. 3 shows HTRF analyses of dose-dependent inhibition of FL-NAH binding to the NSP14 MTase by compounds NSC 111552 and 288387. N=3. TR-FRET values in the presence of compounds were reverse normalized to that of the (−) MTase control (0%) and that of the DMSO control (100%).

FIGS. 4A-4B show analysis of Cytotoxicity and antiviral activity of compounds NSC 111552 and 288387. FIG. 4A shows Cytotoxicity of NSC 111552 (left panel) and 288387 (right panel). Vero cells were treated with various concentrations of NSC 111552 and 288387, followed by cell viability assay at 42 h post-incubation. N=3. FIG. 4B shows Inhibition of SARS-CoV-2 replication by NSC 111552 (left panel) and 288387 (right panel). Vero cells were seeded in 96 well plated. After 24 hours, media was replaced with fresh media containing indicated concentrations of NSC 111552 (left panel) and 288387 (right panel), followed by infection with SARS-CoV-2. At 72 hours post-infection, wells were stained with crystal violet; and viral plaque were counted.

FIGS. 5A-5D show an immunofluorescence assay for detection of SARS-CoV-2 WT and Omicron-infected cells treated with NSC 111552 and 288387. FIGS. 5A and 5C show IFA images of dose-dependent inhibition of SARS-CoV-2 WT Washington Strain (WA) (FIG. 5A) and Omicron BA.1.1 strain (FIG. 5B) by compounds NSC 111552 and 288387. Vero E6 cells were infected with the SARS-CoV-2 WA strain (FIG. 5A and FIG. 5B) and the Omicron strain (FIGS. 5C and 5D), treated with compounds at indicated concentrations for 24 hours for the WA strain or 42 hours for the Omicron strain, fixed and immunolabeled with a primary SARS-CoV-2 nucleocapsid monoclonal antibody and a goat anti-mouse secondary Alexa-488 antibody. Blue, DAPI staining. (FIGS. 5B and 5D) Normalized IFA data shown in FIGS. 5A and 5C. The intensities of Alexa-488 positive cells for the DMSO control were set as 100%. N=3.

FIGS. 6A-6B show synergy between inhibitors of NSP14 MTase, M^{pro}, and RdRp. FIGS. 6A and 6B show dose-response of compounds alone and in combination with fixed concentration of one compound and varying that of the other. N=3.

FIGS. 7A-7B show an analysis of binding of NSC 111552 and 288387 to the SARS-CoV-2 NSP14 protein using MST. FIGS. 7A and 7B show dose-response curves generated by fitting experimental data by titrating NSC 111552 and 288387 from 0.6 mM to 9.1 nM against NSP14 (40 nM). N=3.

FIG. 8 shows a dose-dependent FL-NAH FP assay. FL-NAH (50 nM) was applied to 2-fold diluted concentration series of DENV3, WNV, YFV and ZIKV MTases. FP was calculated by measuring the parallel and perpendicular fluorescence with excitation and emission wavelengths of 485 nm and 528 nm, respectively. N=3.

FIG. 9A shows a dose-dependent inhibition of FL-NAH binding to the DENV3 NS5 MTase by NSC 111552 and 288387 in the presence and absence of DTT (1 mM). N=3.

FIG. 9B shows dose-dependent inhibition of FL-NAH binding to hRNMT by NSC 111552 and 288387. N=3.

FIGS. 10A-10D show HTRF analyses of dose-dependent inhibition of the N7 MTase activity of viral MTases of DENV3 (FIG. 10A), ZIKV (FIG. 10B), WNV (FIG. 10C), and YFV (FIG. 10D) by compounds NSC 111552 and 288387. N=3. HTRF values in the presence of compounds were reverse normalized to that of the (−) MTase control (0%) and that of the DMSO control (100%).

FIG. 11 shows dose-dependent inhibition of Zika-Venus by NSC 111552 and 288387. N=3.

FIG. 12 shows a binding analysis of NSC 111552 and 288387 to the DENV3 NS5 MTase protein using MST.

FIGS. 13A-13D show the SAM-binding sites of MLL1 (FIG. 13A), Ebola virus (FIG. 13B), SARS-CoV-2 NSP14 (FIG. 13C) and Dengue virus (FIG. 13D) MTases.

### DETAILED DESCRIPTION OF THE INVENTION

Any methods, devices, and materials similar or equivalent to those described herein can be used in the practice of this invention. The following definitions are provided to facilitate understanding of certain terms used frequently herein and are not meant to limit the scope of the present disclosure. In the event that there is a plurality of definitions for a term herein, those in this section prevail unless stated otherwise. Headings used herein are for organizational purposes only, and in no way limit, the invention described herein.

As used herein, the terms “subject” and “patient” are used interchangeably. As used herein, a subject can be a mammal such as a non-primate (e.g., cows, pigs, horses, cats, dogs, rats, etc.) or a primate (e.g., monkey and human). In specific embodiments, the subject is a human. The term does not denote a particular age or sex. Thus, adult, and newborn subjects, as well as fetuses, whether male or female, are intended to be included. In one embodiment, the subject is a mammal (e.g., a human) having a disease, disorder, or condition described herein. In another embodiment, the subject is a mammal (e.g., a human) at risk of developing a disease, disorder, or condition described herein. A “patient” is a subject afflicted with a disease or disorder.

The terms “treating” or “treatment” refer to any indicia of success or amelioration of the progression, severity, and/or duration of disease, pathology, or condition, including any objective or subjective parameter such as abatement; remission; diminishing of symptoms or making the injury, pathology or condition more tolerable to the patient; slowing in the rate of degeneration or decline; making the final point of degeneration less debilitating; or improving a patient's physical or mental well-being.

The terms “manage,” “managing,” and “management” refer to preventing or slowing the progression, spread, or worsening of a disease or disorder or of one or more symptoms thereof. In certain cases, the beneficial effects that a subject derives from a prophylactic or therapeutic agent do not result in a cure of the disease or disorder.

The term “effective amount” as used herein refers to the amount of a therapy that is sufficient to reduce and/or ameliorate the severity and/or duration of a given disease, disorder, or condition and/or a symptom related thereto. This term also encompasses an amount necessary for the reduction or amelioration of the advancement or progression of a given disease (e.g., SARS-CoV-2), disorder or condition, reduction or amelioration of the recurrence, development or onset of a given disease, disorder, or condition, and/or to improve or enhance the prophylactic or therapeutic effect(s) of another therapy. In some embodiments, “effective amount” as used herein also refers to the amount of therapy provided herein to achieve a specified result.

As used herein, and unless otherwise specified, the term “therapeutically effective amount” of a composition herein is an amount sufficient to provide a therapeutic benefit in the treatment or management of viral infection or to delay or minimize one or more symptoms associated with the viral infection. A therapeutically effective amount of a composition described herein means an amount of therapeutic agent, alone or in combination with other therapies, which provides a therapeutic benefit in the treatment or management of a viral infection. The term “therapeutically effective amount” can encompass an amount that improves overall therapy, reduces, or avoids symptoms or causes, or enhances the therapeutic efficacy of another therapeutic agent.

The terms “administering,” and “administration” refer to methods of providing a pharmaceutical preparation to a subject. Such methods are well known to those skilled in the art and include, but are not limited to, administering the compositions intranasally, parenterally (e.g., intravenously and subcutaneously), by intramuscular injection, by intraperitoneal injection, intrathecally, transdermally, extracorporeally, topically or the like.

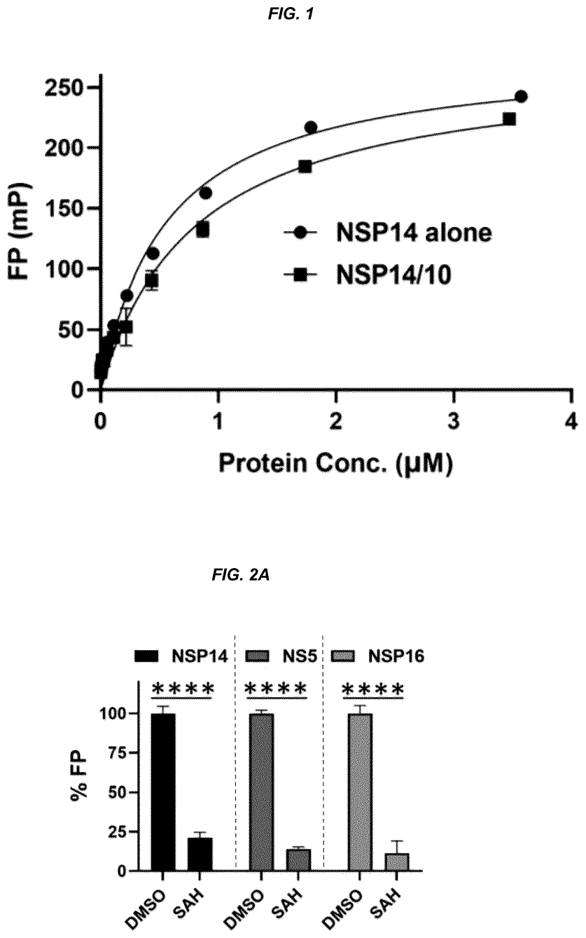

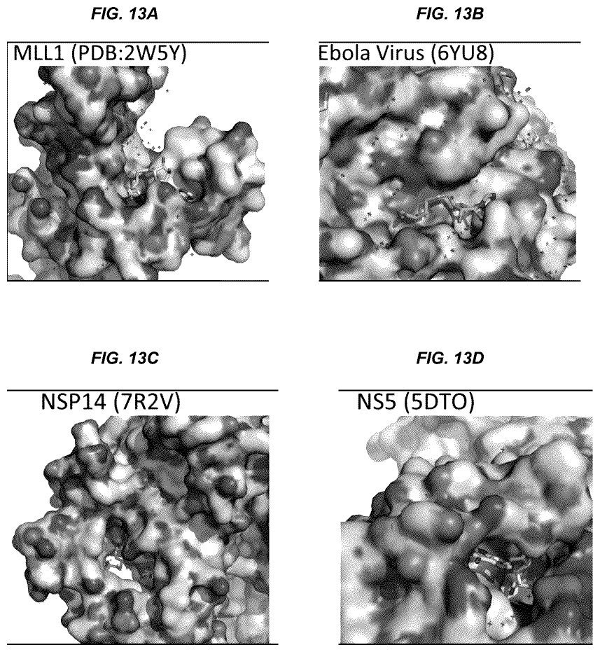

Referring now to FIGS. 1-13, the present invention features methods and compositions for inhibiting viral methyltransferases, specifically for the treatment of a viral infection (e.g., SARS-CoV-2 and flaviviruses). In some embodiments, the present invention features a fluorescence polarization (FP)-based method to identify an inhibitor targeting an S-adenosyl-L-methionine (SAM)-binding site of a viral methyltransferase (MTase) for a coronavirus or a flavivirus, said method comprising: (1) introducing the inhibitor and a fluorescent analog of a methyl donor SAM to the coronavirus or the flavivirus for them to compete to combine with the viral MTase of the coronavirus or the flavivirus; and (2) measuring binding of the fluorescent analog and the viral MTase, wherein binding of the fluorescent analog and the viral MTase increases the fluorescence polarization of the fluorescent analog, wherein binding of the inhibitor and the viral MTase reduces the fluorescence polarization of the fluorescent analog.

In some embodiments, the coronavirus is SARS-CoV-2. In some further embodiments, the fluorescent analog comprises a fluorescent ligand containing fluorescein linked to aza-adenosylhomocysteine. In some embodiments the fluorescent ligand comprises fluorescein N-adenosylhomocysteine (FL-NAH). In some embodiments the fluorescent analog is a non-hydrolyzable fluorescent SAM analog that can mimic SAM to bind SAM-dependent MTases. In some embodiments, the viral MTase comprises SARS-CoV-2 NSP14, SARS-CoV-2 NSP16, flavivirus NS, or a combination thereof.

In some embodiments, the present invention features a composition comprising at least one compound selected from the following:

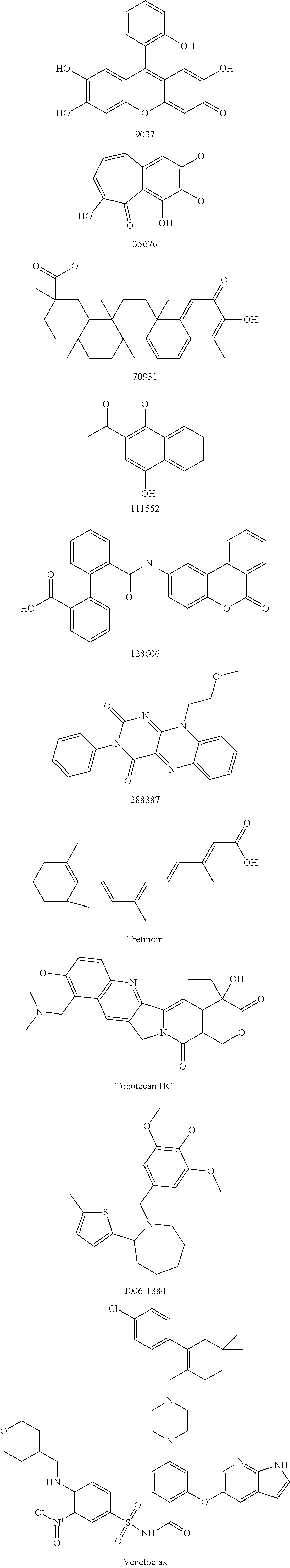

In some embodiments the composition binds at an S-adenosyl-L-methionine (SAM)-binding site of a viral methyltransferase (MTase) for a coronavirus or a flavivirus. In some embodiments the coronavirus is SARS-CoV-2. In some further embodiments, the coronavirus is an omicron strain of SARS-CoV-2. In some embodiments, the coronavirus is another variant strain of SARS-CoV-2, including, but not limited to, alpha, beta, gamma, delta, epsilon, eta, iota, kappa, zeta and mu lineages.

In some embodiments, the compound is 111552. In some embodiments, the compound has a synergistic effect with remdesivir. In other embodiments, the compound has a synergistic effect with other antivirals or COVID drugs. In some further embodiments, the composition may comprise two or more of the compounds. In some embodiments, the composition may compromise three or more compounds. In some further embodiments, the composition may comprise four or more compounds. In some further embodiments, the composition may comprise a combination of five or more of the compounds. In some other embodiments, the composition may comprise 2-4 compounds.

In some embodiments, the present invention features a method of treating a viral infection in a subject in need thereof, the method comprising administering to the subject a therapeutically effective amount of a composition that targets an S-adenosyl-L-methionine (SAM)-binding site of viral methyltransferases (MTases) for a coronavirus or a flavivirus using a SAM as a methyl donor.

In some embodiments the coronavirus is SARS-CoV-2. In some further embodiments the coronavirus is an omicron strain of SARS-CoV-2. In some embodiments the composition comprises at least one compound selected from the following:

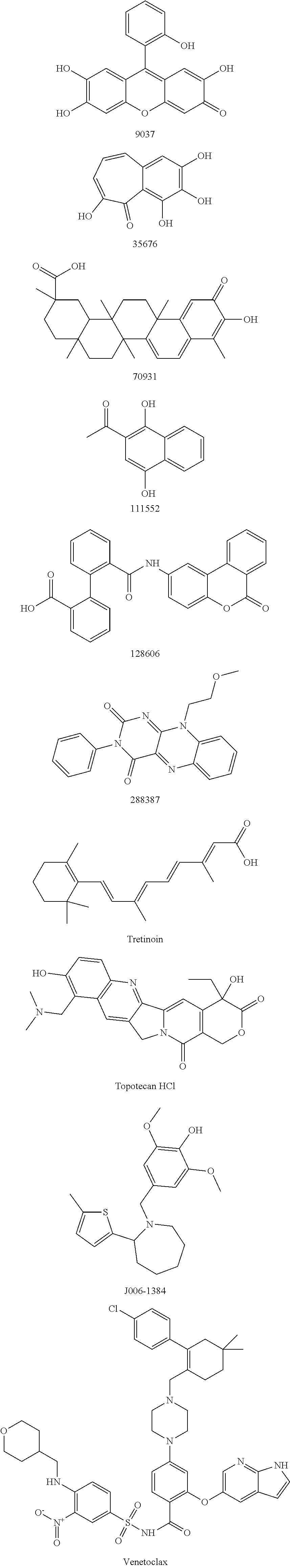

In some further embodiments, the compound is 111552 and has a synergistic effect with remdesivir. In some embodiments, the compound is 111552. In some embodiments, the compound has a synergistic effect with remdesivir. In other embodiments, the compound has a synergistic effect with other antivirals or drugs for treating viral infections.

In some embodiments the flavivirus is dengue virus 1-4, West Nile virus, Zika virus, yellow fever virus, or Japanese encephalitis virus. In some embodiments, the composition binds at the SAM-binding site of the MTases.

In some embodiments, the present invention features a composition for use in treating a viral infection in a subject in need thereof, wherein the composition is selected from a group consisting of:

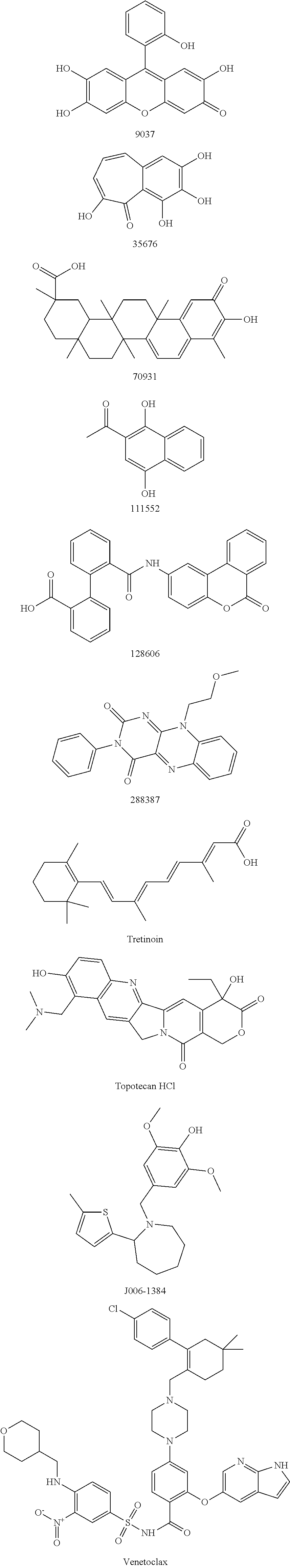

In some embodiments, composition inhibits viral methyltransferases. In some embodiments the composition binds at a S-adenosyl-L-methionine (SAM)-binding site of the viral methyltransferases (MTases). In some embodiments the viral infection is caused by a coronavirus or a flavivirus. In some embodiments the coronavirus is SARS-CoV-2. In some embodiments the coronavirus is an omicron strain of SARS-CoV-2. In some further embodiments the compound is 111552 and has a synergistic effect with remdesivir. In some embodiments, the flavivirus is dengue virus 1-4, West Nile virus, Zika virus, yellow fever virus, or Japanese encephalitis virus.

### Example

The following is a non-limiting example of the present invention. It is to be understood that said example is not intended to limit the present invention in any way. Equivalents or substitutes are within the scope of the present invention.

A fluorescent SAM-analog, FL-NAH was used to develop a fluorescence polarization (FP)-based HTS assay to target reference MTases, the SARS-CoV-2 NSP14, the SARS-CoV-2 NSP16, and flavivirus NS5 MTases, which are essential enzymes for SARS-CoV-2 and flaviviruses to methylate the 5′-cap of viral RNA genome. Pilot screening demonstrated that the HTS assay was very robust and identified several candidate inhibitors, including NSC 111552, and NSC288387, NSC70931, topotecan, and J006-1384. These compounds inhibited the FL-NAH binding to the NSP14, NSP16, and NS5 MTases with low micromolar IC50. Three functional MTase assays were used to unambiguously verify the inhibitory potency of these molecules for the NSP14 and NS5 MTase functions. Binding studies indicated that these molecules bound directly to the NSP14, NSP16, and NS5 MTases with similar low micromolar affinity. Moreover, these molecules significantly inhibited the replication of SARS-CoV-2 and Zika virus in cell-based assays at concentrations not causing significant cytotoxicity. Finally docking suggested that these molecules bind specifically to the SAM-binding site on the NSP14, NSP16, and NS5 MTases. Overall, these molecules represent novel and promising candidates to further develop broad-spectrum inhibitors for management of viral infections.

### HTS Assay.

### FL-NAH FP Assay.

For inhibitor screening against SARS-CoV-2 NSP14, an FP-based assay was performed using FL-NAH, a fluorescent analog of the methyl donor SAM, as described earlier. The screening assay was performed in a reaction buffer consisting of 20 mM HEPES, pH 8.5, 150 mM NaCl, 10% glycerol, 1 mM DTT, 0.01% triton X-100, 50 nM FL-NAH and 0.5 μM NSP14 or NS5. The assay was performed in a 25 μL reaction volume in 96-well black polypropylene plates, against the NCI diversity set VI library (1,584 compounds, 20 plates). NSP14 was initially incubated with DMSO or the inhibitor for 30 min at ambient temperature. FL-NAH was added to the reaction and FP was measured after 30 min using excitation and emission wavelengths of 485 nm and 528 nm, respectively. The FP was indicated in millipolarization units (mP). For IC_{50-disp} measurement, the assay was carried out in the presence of 50 nM FL-NAH and the increasing concentration of the inhibitor or DMSO.

### Viral Inhibition Assay.

To confirm the in vitro inhibitory assay of identified compounds NSC 111552 and 288378 on SARS-CoV-2 and ZIKV replication, Vero cells (ATCC #CCL-81) were used that are a model cell line for SARS-CoV-2 and ZIKV viral infection assay. SARS-Related Coronavirus 2 (SARS-Cov-2), Isolate USA-WA1/2020 (BEI NR-52281), was obtained from a laboratory. Vero cells were suspended in the DMEM medium containing 10% FBS and seeded 3.125×10⁴ cells into each well of 96 wells plates and cultured overnight with 5% CO₂ at 37° C. The next day with 90% of cell confluency, a 2× solution was generated by dispensing 10 mM stocks of compounds into a V-bottom 96-well plate and DMSO control. The spent media were removed from assay plate culturing Vero cells. 100 μl of fresh medium with compound and SARS-CoV-2 virus stock with proper dilution to reach about 137 virus plaques per well was added to each well. The plates were further incubated for 3 hours with 5% CO₂ at 37° C., then overlayed with 100 μl per well of 1% methylcellulose in media. Plates were further incubated with 5% CO₂ at 37° C. for 72 hrs. After 72 hours post-infection, plates were fixed with 10% Neutral Buffered Formalin for 30 minutes and stained for SARS-CoV-2 viral plaque using 0.9% crystal violet. All SARS-CoV-2 related work was conducted in a biological safety cabinet in a biosafety level 3 laboratory at the University of Arizona. The plaque was counted, and EC₅₀ was calculated using GraphPad Prism 9.

### Immunofluorescence Assay.

Vero E6 cells were seeded at a density of 3×10⁴ cells per well in a 96-well flat bottom plate and incubated at 37° C. for 24 hours. After addition of compounds at different concentrations, the cells were infected with the SARS-CoV-2 WT strain (Washington strain, 0.01 MOI) or the Omicron strain (BA.1.1, 0.05 MOI), the cells were incubated for 24 hours for the Washington strain or 42 hours for the Omicron strain.

Cells were fixed with 10% 10% Formalin Solution for 30 minutes. Cells were detected using 1:100-diluted SARS-CoV-2 nucleocapsid monoclonal antibody (Invitrogen, MA17403) at 4° C. for overnight. After washing with double-distilled water, cells were incubated with a 1:2000 dilution of goat anti-mouse IgG (H+L) highly cross-adsorbed secondary antibody, Alexa Fluor™ Plus 488 (Thermo Scientific, A32723TR) and counterstained with 1 μg of 4′,6-diamidino-2-phenylindole (DAPI) per ml. Cells were visualized using a microscope.

### Compound Combination.

The 2D compound-compound interactions were determined by checkerboard titration in a 96-well plate. Vero-E6 cells were seeded one day before combination test at 3×10⁴ cells/each well. The first compound (A) was serially diluted along the x axis (columns 3 to 8); and the second compound (B) was serially diluted along the y axis (from row A to F) in the 96-well plate. The last two columns (columns 9 and 10) contained compound B-alone controls and the last two rows (row G and H) contained compound A-alone controls. After the cells grew into monolayer, the medium was discard, followed by addition of 50 μl compound mixture. Then 50 μl SARS-CoV-2 WA strain was added at MOI of 0.01.

The plate was incubated at 37° C., 5% CO₂ for 1 hour. Then 100 μl overlay medium (DMEM+2% FBS+1% Methylcellulose) was added and the plate was further incubated at 37° C., 5% CO₂. After 3 days, cells were fixed with 10% neutral formalin for 30 minutes, wash with water for more than 6 times and then stained with 50 μl Crystal Violet (0.5% in methanol/water) for 4 minutes. The staining solution was removed by aspiration and the plate was rinsed with water, and dried thoroughly. Plaques were counted and numbers were recorded to calculate the percentage of inhibition. SynergyFinder was used to analyze the synergistic effect of the two compounds (https://synergyfinder.fimm.fi/synergy/20230202071113839465/). Each combination testing experiment has three replicates.

FL-NAH Binds to the NSP14 MTase and NSP14/10 Complex with Similar Binding Affinity.

Using FL-NAH, we developed an FP-based assay to identify and characterize inhibitors targeting the co-factor SAM-binding site of the SARS-CoV-2 NSP14 MTase. FL-NAH is a fluorescent non-hydrolyzable fluorescent SAM analog that can mimic SAM to bind SAM-dependent MTases. It was previously used to develop an HTS SAM-displacement assay for a histone MTase MLL1. The fluorescent ligand containing fluorescein linked to aza-adenosylhomocysteine—namely fluorescein N-adenosylhomocysteine (FL-NAH)—is built based on the backbone structure of the SAM cofactor.

Using purified proteins, we investigated whether binding of FL-NAH to the NSP14 MTase could be monitored by FP. As shown in FIG. 1, FL-NAH could bind to the NSP14 MTase, leading to FP changes in a manner depending on the concentration of NSP14. By fitting the experimental data, we determined that FL-NAH bound to the NSP14 MTase with a binding affinity K_{D} of 0.56 μM. In addition, we investigated if FL-NAH is also bound to the NSP14/NSP10 complex. Our results showed that the FL-NAH also bound to the NSP14-NSP10 complex dose-dependently, with a slightly weaker binding affinity of 0.81 μM than that for NSP14 alone (FIG. 1). The results confirmed that NSP10 was not required for the NSP14 MTase function. Since NSP10 had only minimal effects on the NSP14 MTase activity, all the following experiments were performed using the NSP14 MTase alone.

### FP-Based FL-NAH-Displacement Assay is Universal for SAM-Dependent MTases.

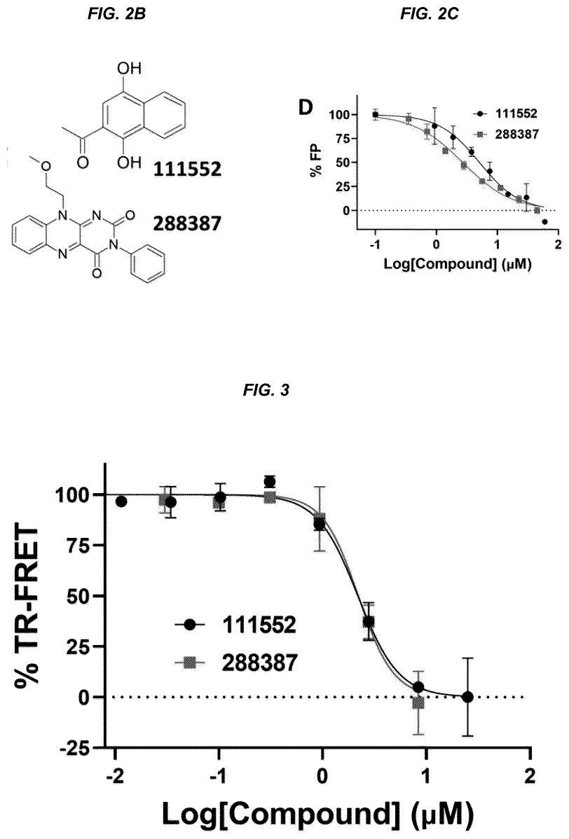

Our results showed that the SAM-displacement FP assay is very robust in a 96-well plate format with satisfactory signal/background (S/B) ratio (4.7), Z-factor (0.7), and coefficient variation (CV, 4.5%) against the NSP14 MTase (FIG. 2A). The optimized HTS assay contained 50 nM FL-NAH and 0.5 μM NSP14 in a 25 μL reaction mixture. Clearly, FL-NAH binding to NSP16 and the ZIKV NS5 MTases led to significant FP, which could be quenched by SAH or SAM (FIG. 2A). These data demonstrated that the FL-NAH assay could be universally applicable to any viral MTases that use SAM as a methyl donor.

### HTS Screening

To identify inhibitors targeting the SAM-binding site of the SARS-CoV-2 NSP14 MTase, we performed a small scale HTS against the NCI diversity set VI compound library, containing 1,584 compounds dissolved in DMSO in twenty 96-well plates. A single concentration of 15 μM of each compound was used in HTS. For each plate screening, DMSO was used as a negative control, whereas SAH at 25 μM was used as a positive control for inhibition. The quality of the screening assay was assessed by calculating Z-factor, S/B ratio, and CV for each plate, average of which are 0.8, 4.0 and 3%, respectively. The results indicated a high-quality screen.

The FL-NAH-displacement primary HTS screen identified 12 compounds showing inhibition larger than 50% for the binding of FL-NAH to the NSP14 MTase. Ten compounds were eliminated from further investigation due to compound autofluorescence and upon cheminformatics analysis of the chemical structures of the hit compounds. Two compounds, NSC 111552 and 288387 were chosen for further dose-response confirmation (FIG. 2B). Our results showed that NSC 111552 and 288387 inhibited the FL-NAH binding to the SARS-CoV-2 NSP14 MTase dose-dependently with IC_{50-disp} values of 5.1 μM and 2.7 μM, respectively (FIG. 2C, Table 1).

TABLE 1
Inhibition of FL-NAH binding to the SARS-CoV-2 NSP14 MTases (IC_{50-disp}),
inhibition of MTase activity (IC_{50-HTRF}, IC_{50-TLC}, IC_{50-MS}), antiviral efficacy
(EC₅₀), and cytotoxicity (CC₅₀) in Vero cells of compounds.

| | FL-NAH | NSP14 MTase activity | | | Antiviral | Cytotoxicity | NSP14 |
|---|---|---|---|---|---|---|---|
| | Inhibition (μM) | | | | | | |
| NSC | IC_{50-disp} | IC_{50-HTRF} | IC_{50-TLC} | IC_{50-MS} | EC₅₀ | CC₅₀ | K_{D} |
| 111552 | 5.1 | 2.2 | 7.0 | 1.5 | 8.5 | 61.8 | 2.1 |
| 288387 | 2.7 | 2.2 | 2.5 | 4.3 | 5.7 | 64.4 | 1.5 |

### Inhibition of the MTase Activity.

Using a GpppA dinucleotide as a substrate as described previously, we performed the HTRF functional MTase assay, using the EPIgeneous Methyltransferase kit (Cisbio, MA). Our result showed that compounds NSC 111552 and 288387 nearly equally inhibited the NSP14 N7-MTase activity with IC50-HTRF values of 2.2 μM (FIG. 3, Table 1).

### Cytotoxicity and Antiviral Analyses.

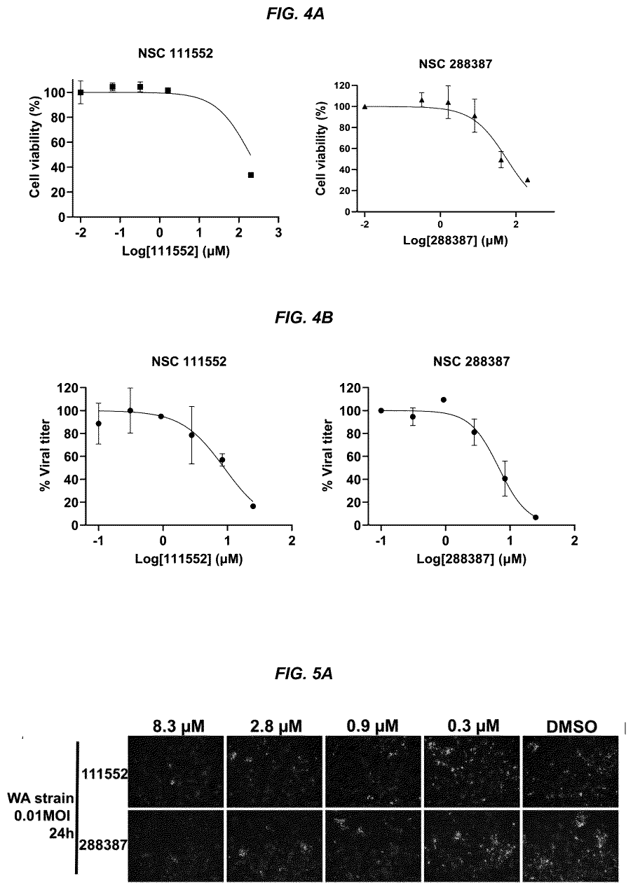

To confirm the inhibitory effects of viral replication in mammalian cells, we first performed a cell proliferation assay to measure the cytotoxicity of these compounds in Vero cells. A WST-8 cell proliferation assay showed that the two compounds, NSC 111552 and 288378, are not toxic to mammalian cells with CC₅₀ of 61.8 μM and 64.4 M, respectively (FIG. 4A, Table 1).

Viral titer reduction assay was performed to evaluate the compounds' antiviral efficacy. The SARS-CoV-2 viral titer was reduced in a dose-dependent manner by compounds NSC111552 and NSC288378 (FIG. 4B, Table 1). NSC 111552 and NSC 288378 showed an antiviral efficacy EC₅₀ of 8.5 μM and 5.7 μM, respectively (Table 1).

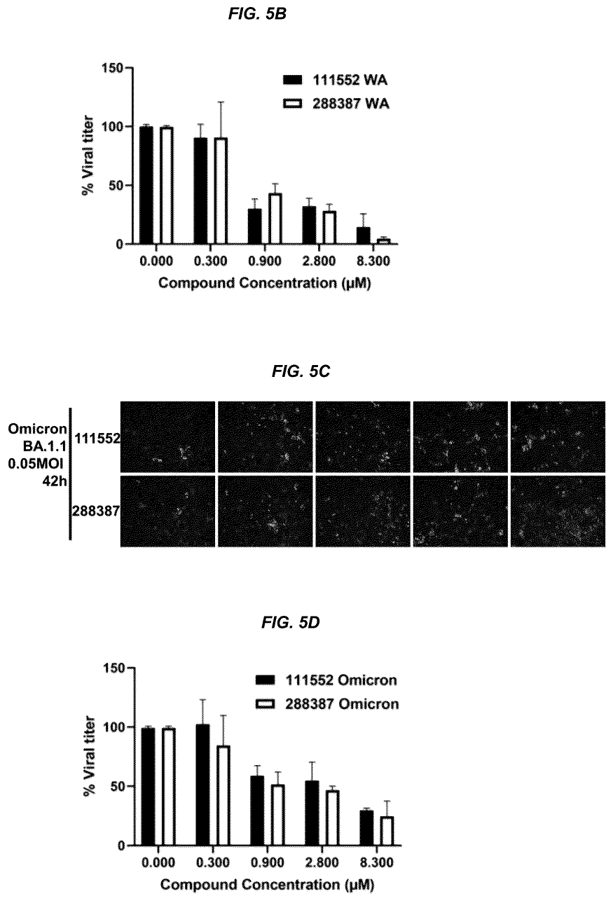

We also performed the immunofluorescence assay (IFA) in both SARS-CoV-2 wild-type (WT) strain (Washington strain, 0.01 MOI) or Omicron strain (BA.1.1, 0.05 MOI). Our results suggested that the compounds NSC111552 and NSC288378 dose-dependently reduced the viral titer (FIGS. 5A-5D). Overall, these results indicated that the compounds we identified have antiviral activity in mammalian cells with limited cytotoxic effects.

Combination with Drugs.

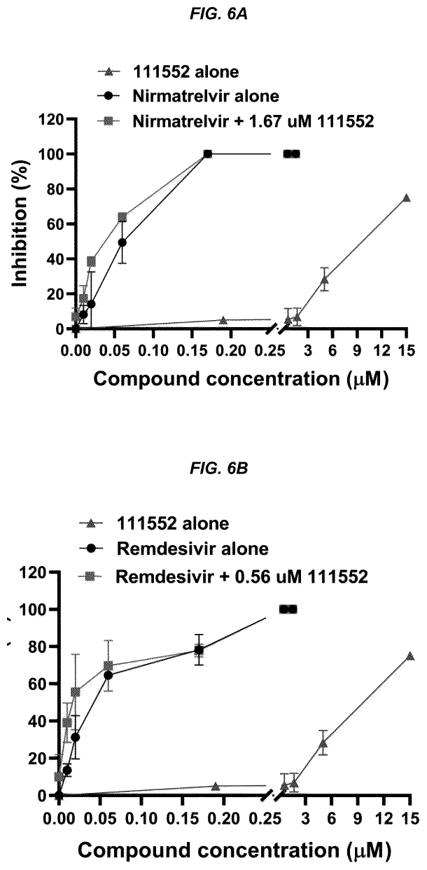

We explored the potential of our NSP14 MTase inhibitor NSC111552 in combination with known SARS-CoV-2 drugs, including nirmatrelvir and remdesivir, targeting the SARS-CoV-2 main protease (M^{pro}) and RNA-dependent RNA polymerase (RdRp), respectively. We conducted a checkerboard combination assay as we and other described previously. Our results showed that these inhibitors targeting different SARS-CoV-2 enzymes have significant synergistic effects, with maximal ZIP-scores of 21 and 24 between MTase inhibitor NSC111552 and M^{pro} inhibitor nirmatrelvir, and between NSC111552 and RdRp inhibitor remdesivir, respectively. In the presence of NSC111552 (1.67 M), the EC₅₀ for nirmatrelvir was shifted ˜1.8-fold, from 56 nM to about 31 nM (FIG. 6A). Similarly, NSC111552 (0.56 μM) decreased EC₅₀ for remdesivir about 2.4-fold, from 40 nM to 17 nM (FIG. 6B). It is noted that NSC111552 at these concentrations only minimally impacted SARS-CoV-2 replication (<10% inhibition). Therefore, these results suggest that the NSP14 MTase inhibitor synergizes with M^{pro} and RdRp inhibitors.

### Direct Binding of NSC111552 and NSC288378.

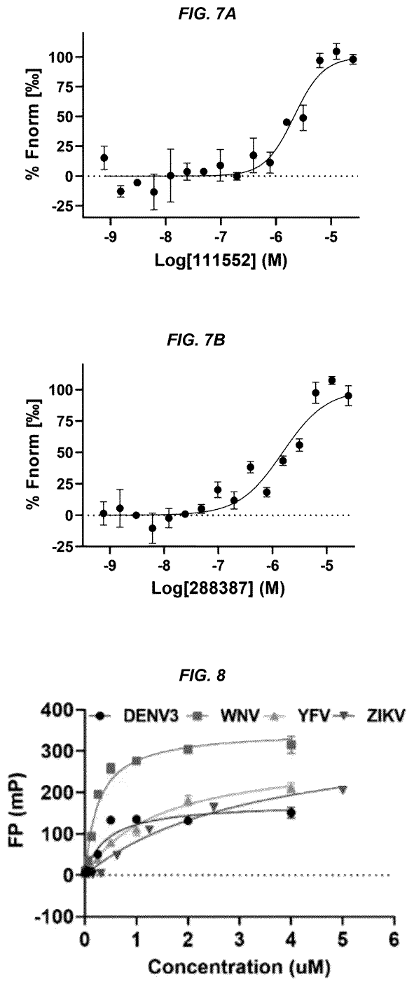

We labeled the target protein NSP14 with a fluorescent dye according to manufactory manual. NSC 111552 and 288378 bound to NSP14 with a binding constant (K_{D}) of 2.1 μM and 1.5 μM, respectively. These data confirmed direct binding of NSC 111552 and 288378 to the NSP14 MTase (FIGS. 7A-7B).

### Compositions.

### FL-NAH FP Assay.

An FP-based assay using FL-NAH, a fluorescent analog of the methyl donor SAM, was carried out for inhibitor screening against DENV3 NS5. The reaction solution used for the screening assay was composed of 20 mM HEPES, pH 8.5, 50 mM NaCl, 10% glycerol, 1 mM DTT, 0.1% triton X-100, 50 nM FL-NAH, and 0.5 μM DENV3 NS5. The NCI Diversity Set VI library was used in the assay, which was conducted in a 25 μL reaction volume in 96-well black polypropylene plates (1584 compounds, 20 plates). In the beginning, DENV3 NS5 was incubated with DMSO or the inhibitor for 30 min at room temperature. After 30 min, FP was determined by adding FL-NAH to the reaction and utilizing 485 and 528 nm for excitation and emission, respectively. Millipolarization (mP) units were used to define FP. To determine IC50-disp defined as 50% inhibition of FP resulting from FL-NAH binding to the DNEV3 NS5 MTase by a compound, the assay was done with 50 nM FL-NAH and a concentration series of the inhibitor or DMSO.

### HTRF MTase Functional Assay.

By observing the release of SAH from the MTase activity, the DENV3 NS5 activity was determined. A commercially available MTase kit called EPIgeneous Methyltransferase Assay 1000 Tests was used to assess the MTase reaction product SAH (CisBio Bioassays). As previously described, the MTase reaction was carried out at 30° C. in a 10 μL reaction volume using a 100 nM DENV3 NS5 protein, 2 mM SAM (CisBio), and 2 mM capped Gppp-RNA in a P7 reaction buffer composed of 50 mM Tris-HCl, pH 7.0, 2 mM DTT, and 20 mM NaCl·24 The DENV3 NS5 was added to a final concentration of 1 μM to start the reaction, which was then allowed to run for 20 min before being quenched by adding 2 μL of 5 M NaCl to a final concentration of 1 M.

After quenching, 2 μL of detection Buffer 1 (CisBio) was added to the reaction mixture and incubated for 10 min. Following 10 min of incubation, 4 μL of the diluted SAH d2 reagent (CisBio) was added. After 5 min, 4 μL of diluted SAH Tb Crypate antibody solution was added to the reaction mixture and further incubated with shaking at 500 rpm for an hour. Homogeneous time-resolved fluorescence (HTRF) measurements were taken on a BioTek Cytation 5 microplate reader. To take a reading following the excitation at 330 nm, a lag time of 100 us was utilized. The readings were taken at emission wavelengths of 665 and 620 nm, respectively. The experimental HTRF ratio (HTRFexp) was calculated as a ratio of emission intensities: 1665/1620. The HTRF ratio was determined using wells devoid of the enzyme (E0) and the SAH-d2 molecule (d20), which stand for the highest and minimum HTRF values, respectively, in order to determine the normalized HTRF ratio. A linear transformation of the experimental HTRF ratio, the E0 ratio, and the d20 ratio was then used to determine the normalized HTRF ratio (Equation 1).

$$Normalized HTRF = \frac{H T R {F}_{exp} - d {2}_{0}}{{E}_{0} - d {2}_{0}} \qquad Equation 1$$

### Cytotoxicity Assay.

As previously mentioned, cytotoxicity to the human lung carcinoma A549 cells was assessed using a WST-8 cell proliferation assay kit (Dojindo Molecular Technologies, Inc.).

### Antiviral Test Against ZIKA Virus.

To evaluate the antiviral potency of NSC 111552 and 288378, we seeded the Vero cells (2×10⁶) in each well of a 96 well plate. After 24 h of incubation at 37° C. with 5% CO₂, cells were infected with a full length infectious ZIKA clone expressing Venus fluorescent protein (ZIKA-Venus)⁶⁷ at MOI of 1 and cells were treated with 3-fold serial dilution of NSC 111552 and 288378. We kept DMSO as a negative control. After 5 dpi, cells were washed 3 times with PBS and images were taken with the Cytation 5 imaging reader (BioTek) using GFP channels via 10× objective lens and were analyzed with the Gen5 3.10 software (BioTek). The GFP expressing cells was counted, and GraphPad Prism 9 was used to determine the EC₅₀.

### Binding of FL-NAH to Flavivirus MTase.

We performed an FP assay to monitor FL-NAH binding to these representative flaviviral MTases. Our result indicated that significant FP increases were observed for FL-NAH binding to flaviviral MTases with increasing MTase concentrations, suggesting that FL-NAH binds to these flaviviral MTases dose-dependently (FIG. 8). By fitting an experimental curve, we determined the binding affinity (KD) of FL-NAH to these MTases (Table 2). FL-NAH binds the WNV and DENV3 MTases with a high affinity of 0.28 and 0.61 μM, respectively. The YFV and ZIKV MTases bind FL-NAH with a moderate affinity of 3 and 3.5 M, respectively.

TABLE 2
Binding affinity of FL-NAH to representative flaviviral MTases.

| | DENV3 | WNV | YFV | ZIKV |
|---|---|---|---|---|
| K_{D} (μM) | 0.61 | 0.28 | 3 | 3.5 |

### FP-Based FL-NAH-Displacement HT Screening.

Using SAH (25 μM) as a control inhibitor, we evaluated the suitability of the FL-NAH-displacement assay for HTS in a 96-well plate format. Our findings demonstrated that the FL-NAH displacement FP assay is well suitable for HTS, with a satisfactory Z′-factor of 0.87, high signal/background (S/B) ratio of 7.1, and low coefficient of variation (CV) of 2.1. These parameters satisfy or are better than the guideline criteria.

To find compounds that target the SAM-binding site of the DENV NS5 MTase, we carried out a small-scale HTS against the NCI Diversity Set VI compound library, composed of 1584 small molecules. For HTS, each compound was applied at a single concentration of 15 μM. We used SAH at a concentration of 25 M as an inhibitory positive control, and DMSO as a negative control for each plate. The Z′-factor, S/B ratio, and CV for each plate were calculated to determine the quality of the screening assay, with average values of 0.79, 3.96, and 3.1%, respectively. The outcomes suggested a screen of excellent quality.

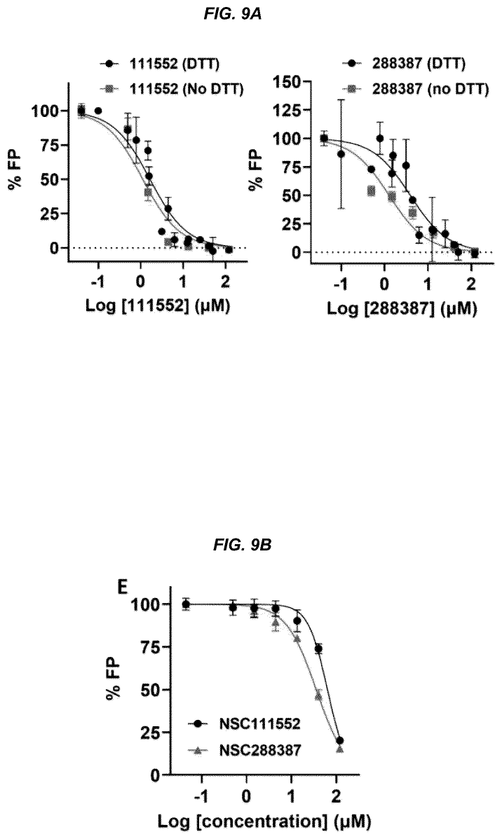

Primary HTS screen identified 20 compounds showing inhibition of more than 60% at 15 μM. Eight compounds were discarded due to compound autofluorescence, fluorescence quenching, or bad chemical properties predicted from the chem informatic analysis. 12 compounds were reordered and subjected to the dose-response inhibition of FL-NAH binding to the DENV3 MTase. Two compounds, including NSC 111552 and 288387, showed dose-dependent inhibition of FL-NAH binding to the DENV3 NS5 MTase with IC50-disp values of 0.98 and 4.2 M, respectively (FIG. 9A and Table 3).

TABLE 3
Inhibition of FL-NAH binding to representative MTases.

| | IC_{50-disp} (μM) | | | | | |
|---|---|---|---|---|---|---|
| | No DTT | DTT (1 mM) | | | | |
| MTases | DENV3 | DENV3 | ZIKV | WNV | YFV | hRNMT |
| 111552 | 1.1 | 1.6 | 7.3 | 5.7 | 46 | 63.3 |
| 288387 | 1.3 | 3.9 | 4.9 | 13 | 10 | 35.5 |

### Compound Liability.

To address possible pan-assay interference compound (PAINS) properties of the hits, compounds NSC 111552 and 288387 were screened for the PAINS properties using two in silico methods. First, chemical structures were run using the FAF4-Drugs script via a web portal of the Parisian Resource in Structural Bioinformatics. Both compounds were screened using the three available PAINS filters A, B, and C. In each case, compounds NSC 111552 and 288387 passed. Next, structures of NSC 111552 and 288387 were screened through the PAINS-Remover program. Our results showed that both compounds NSC 111552 and 288387 passed in silico screening in PAINS Remover.

The two compounds identified through HTS appear to be redox cyclers that generate hydrogen peroxide (H2O2) in the presence of strong reducing agents such as dithiothreitol (DTT) used in nearly all of our biochemical assays. For redox cycling compounds, the observed inhibitory activity could have resulted from the promiscuous artifacts by generated H2O2.

To rule out this possibility, we repeated the FP assay for these two compounds in the presence and absence of DTT (FIG. 9A and Table 3). Our findings indicated that NSC111552 had similar potency in the inhibition of FL-NAH binding to the DENV3 MTase with or without DTT (Table 3). DTT had a slight effect on the activity of NSC288387, reducing its potency by about threefold in inhibiting FL-NAH binding to the DENV3 MTase in the presence of 1 mM DTT compared to its potency in the absence of DTT. The results indicated that the redox cycling potential of NSC288387 did not affect NSC288387's inhibitory activity, as the addition of DTT did not lead to a lower IC50-disp value compared to that without DTT, which would have indicated an opposite result. Overall, the results suggest that the redox property of the compounds does not affect the inhibitory activity of these compounds on FL-NAH binding to the DENV3 MTase in the assay (FIG. 9A and Table 3).

### NSC 111552 and 288387 are Specific to Viral MTases.

Inhibitors targeting the SAM-binding site of viral MTases could also affect human MTases, as SAM is the common methyl donor for nearly all MTases. To further investigate the specificity of these compounds, we tested the NSC 111552 and 288387 compounds against a representative Human RNA MTase (hRNMT), as we did previously. Our result showed that the IC50-disp values for inhibition of hRNMT by NSC 111552 and 288387 were 63.3 and 35.5 μM, respectively (FIG. 9B and Table 3). These values are significantly higher than those for viral MTases of DENV3, WNV, and ZIKV, and also slightly higher than that for the YFV MTase. The results suggest that NSC 111552 and 288387 exhibit varying selectivity toward viral and human MTases. NSC111552 shows greater specificity toward the MTases of DENV3, ZIKV, and WNV compared to hRNMT. However, it lacks selectivity against the YFV MTase and hRNMT. On the other hand, NSC288387 displays higher specificity for the MTases of DENV3 and ZIKV compared to hRNMT, although the selectivity between the viral MTases (WNV and YFV) and hRNMT is comparatively lower.

### Inhibition of the N7 MTase Activity.

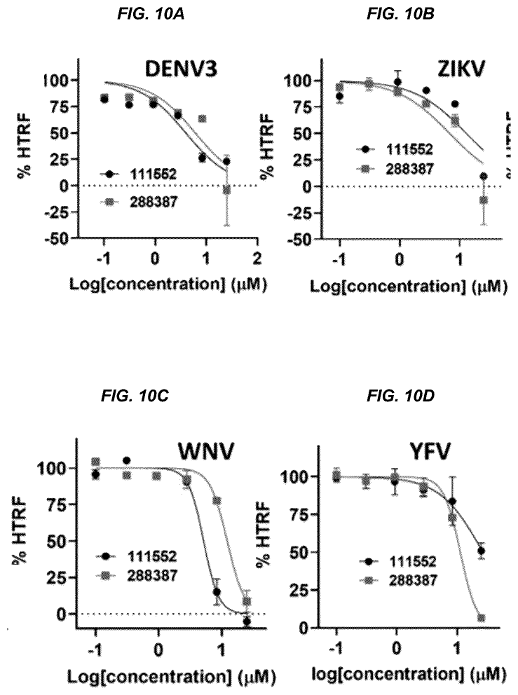

Using this HTRF function assay, we measured the inhibitory efficacy of hit compounds in the inhibition of the methyl transfer activity of viral MTases of DENV3, ZIKV, WNV, and YFV. As shown in FIGS. 10A-10D, compounds NSC 111552 and 288387 dose-dependently inhibited the viral MTase activity with IC50-HTRF values in a low micromolar range (Table 4).

TABLE 4
Characterization of identified MTase inhibitors.

| | | | | | IC_{50-TLC} | K_{D} | EC_{50-DENV2} | | EC_{50-ZIKV} | | |
|---|---|---|---|---|---|---|---|---|---|---|---|
| | IC_{50-HTRF} (μM) | | | | (μM) | (μM) | (μM) | | (μM) | | CC₅₀ |
| | DENV3 | ZIKV | WNV | YFV | DENV3 | DENV3 | Replic | IFA | PRA | IFA | (μM) |
| 111552 | 3.9 | 12.9 | 5.2 | 26.7 | 1.1 | 4.6 | 5.0 | 0.32 | 1.4 | 0.90 | 61.8 |
| 288387 | 6.0 | 6.8 | 12.2 | 11.2 | 1.6 | 3.2 | 11.4 | 0.53 | 0.2 | 0.69 | 64.4 |

### Cytotoxicity and Antiviral Analyses.

To further characterize these candidate inhibitors, we investigated the cytotoxicity of NSC 111552 and 288387 to human A549 lung carcinoma cells, using a WST-8 cell viability kit, as we described previously. Our results showed that NSC288387 was moderately toxic to the A549 cells with a cytotoxicity CC50 of 57 μM, whereas NSC111552 was not toxic to the A549 cells until a very high concentration, with CC50 estimated as 187 μM (Table 4).

Next, we carried out a cell-based replicon study to investigate if NSC 111552 and 288387 could lower viral replication in cell culture. Our results showed that NSC 111552 and 288387 dose-dependently inhibited DENV2 replication using a replicon (Replic) cell line of DENV serotype 2 (DENV2) (Table 4). The EC50 values for NSC 111552 and 288387 for DENV2 were found to be 5.0 and 11.4 μM, respectively.

We further carried out an antiviral immunofluorescence assay (IFA) to investigate the antiviral efficacy of these two inhibitors against DENV2 and ZIKV. We showed that NSC 111552 and 288387 were potent inhibitors against both DENV2 and ZIKV (Table 4).

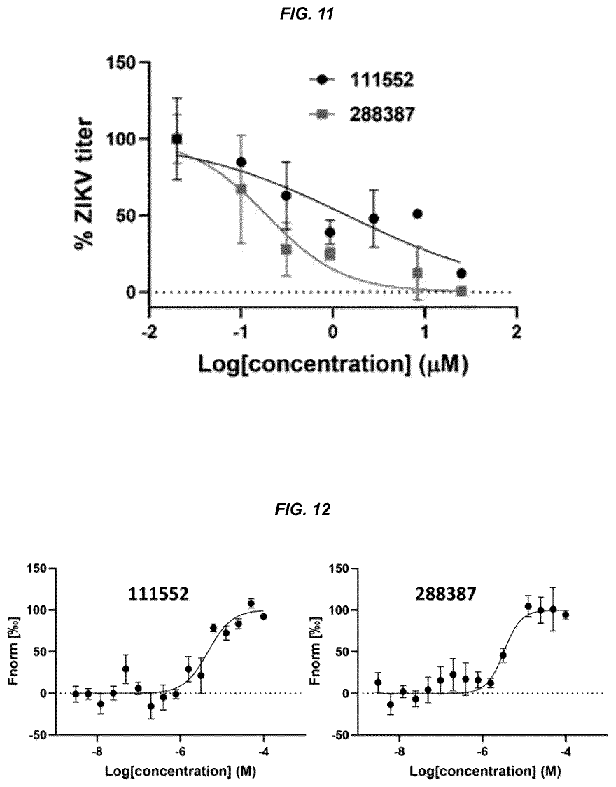

We next carried out an antiviral plaque reduction assay (PRA) to investigate the antiviral efficacy of these two inhibitors against ZIKV. By using a Venus-expressing ZIKV we generated previously, we showed that NSC 111552 and 288387 were potent inhibitors against ZIKV, with EC₅₀ values of 1.4 and 0.2 μM, respectively (FIG. 11 and Table 4).

### Direct Binding of NSC 111552 and 288387 to the DENV3 NS5 MTase.

By using microscale thermophoresis (MST), we investigated if NSC 111552 and 288387 are directly bound to the DENV3 NS5 MTase protein. Our results showed that NSC 111552 and 288387 dose-dependently bound to the DENV3 NS5 MTase with a binding affinity (KD) of 4.6 and 3.2 μM, respectively (Table 4). These data confirmed the direct binding of NSC 111552 and 288387 to the DENV3 NS5 MTase (FIG. 12).

As used herein, the term “about” refers to plus or minus 10% of the referenced number.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

1. A fluorescence polarization (FP)-based method to identify an inhibitor targeting an S-adenosyl-L-methionine (SAM)-binding site of a viral methyltransferase (MTase) for a coronavirus or a flavivirus, said method comprising:
a. introducing the inhibitor and a fluorescent analog of a methyl donor SAM to the coronavirus or the flavivirus for them to compete to combine with the viral MTase of the coronavirus or the flavivirus; and
b. measuring binding of the fluorescent analog and the viral MTase,
wherein binding of the fluorescent analog and the viral MTase increases the fluorescence polarization of the fluorescent analog,
wherein binding of the inhibitor and the viral MTase reduces the fluorescence polarization of the fluorescent analog.

2. The method of claim 1, wherein the coronavirus is SARS-CoV-2.

3. The method of claim 1, wherein the fluorescent analog comprises a fluorescent ligand containing fluorescein linked to aza-adenosylhomocysteine.

4. The method of claim 3, wherein the fluorescent ligand comprises fluorescein N-adenosylhomocysteine (FL-NAH).

5. The method of claim 1, wherein the fluorescent analog is a non-hydrolyzable fluorescent SAM analog that can mimic SAM to bind SAM-dependent MTases.

6. The method of claim 1, wherein the viral MTase comprises SARS-CoV-2 NSP14, SARS-CoV-2 NSP16, flavivirus NS, or a combination thereof.

7. A composition comprising at least one compound selected from the following:

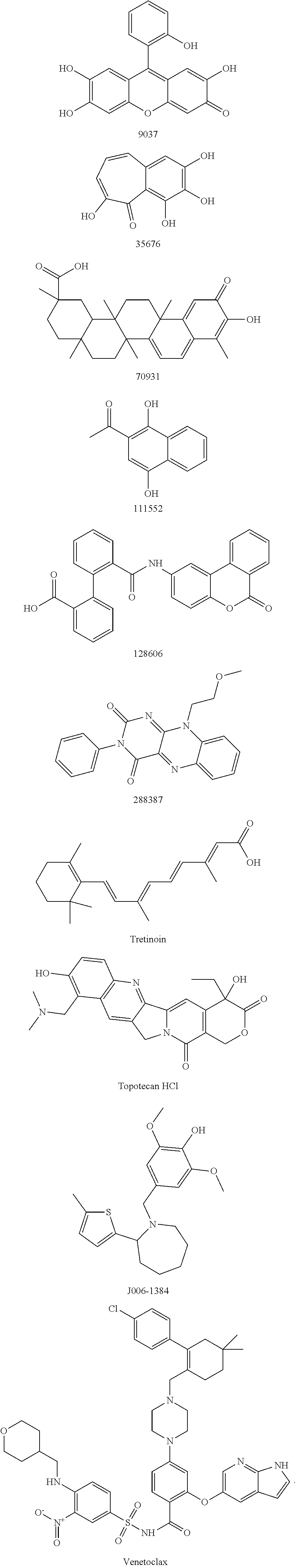

8. The composition of claim 7, wherein the composition binds at an S-adenosyl-L-methionine (SAM)-binding site of a viral methyltransferase (MTase) for a coronavirus or a flavivirus.

9. The composition of claim 8, wherein the coronavirus is SARS-CoV-2.

10. The composition of claim 9, wherein the coronavirus is an omicron strain of SARS-CoV-2.

11. The composition of claim 10, wherein the compound is 111552 and has a synergistic effect with remdesivir.

12. A method of treating a viral infection in a subject in need thereof, the method comprising administering to the subject a therapeutically effective amount of a composition that targets an S-adenosyl-L-methionine (SAM)-binding site of viral methyltransferases (MTases) for a coronavirus or a flavivirus using a SAM as a methyl donor.

13. The method of claim 12, wherein the coronavirus is SARS-CoV-2.

14. The method of claim 13, wherein the coronavirus is an omicron strain of SARS-CoV-2.

15. The method of claim 12, wherein the composition comprises at least one compound selected from the following:

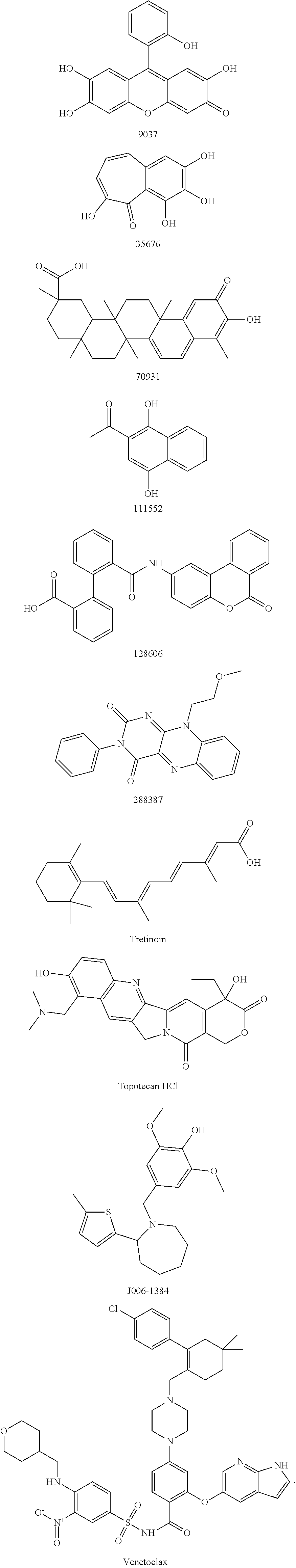

16. The method of claim 14, wherein the compound is 111552 and has a synergistic effect with remdesivir.

17. The method of claim 12, wherein the flavivirus is dengue virus 1-4, West Nile virus, Zika virus, yellow fever virus, or Japanese encephalitis virus.

18. The method of claim 12, wherein the composition binds at the SAM-binding site of the MTases.

19-26. (canceled)
